(19) United States
(12) Patent Application Publication
(10) Pub. No.: US 2012/0302945 A1
(43) Pub. Date: Nov. 29, 2012

(54) METHOD AND APPARATUS FOR THE DETERMINATION OF GAS IN A FLUID PUMPED THROUGH A PUMPING DEVICE

(75) Inventors: Frank HEDMANN, Volkach (DE); Torsten Hochrein, Knetzgau (DE)

(73) Assignee: corporation Fresenius Medical Care Deutschland GmbH, Bad Homburg (DE)

(21) Appl. No.: 13/478,382

(22) Filed: May 23, 2012

Related U.S. Application Data
(60) Provisional application No. 61/491,437, filed on May 31, 2011.

(30) Foreign Application Priority Data
May 27, 2011 (DE) 10 2011 105 824.2

(51) Int. Cl. G01N 7/00; A61M 1/28

(52) U.S. Cl. 604/29; 73/19.05

(57) ABSTRACT

This invention relates to a method for the determination of gas in a fluid, wherein the mixture of gas and fluid both is delivered by a pump and is subjected to a change in volume and pressure, operating parameters of the pump, which represents the change in volume and pressure, are detected, and from the operating parameters of the pump the gas content is determined in consideration of a system compressibility, wherein the system compressibility in the pumping device filled with gas is determined by sequence of process steps. The invention furthermore relates to an apparatus for performing this method into a dialysis machine which contains a corresponding apparatus.

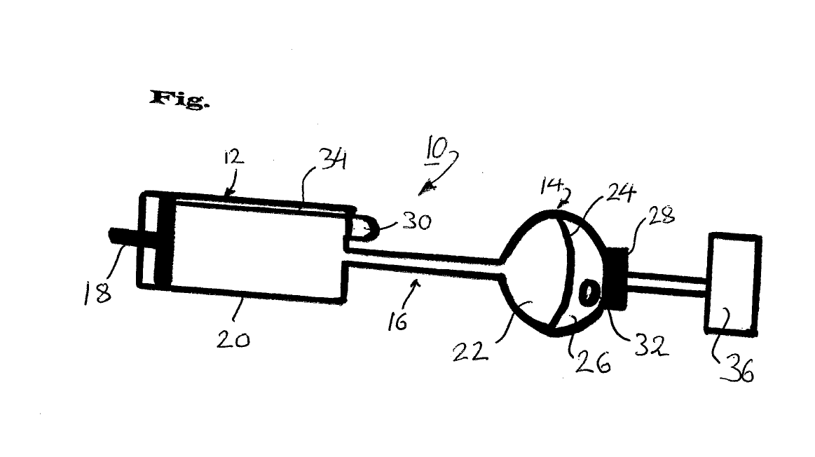

This invention relates to a method and an apparatus for the determination of gas in a fluid pumped through a pumping device.

When delivering, balancing and dosing medical fluids, it is important to detect the gas content in the fluid transported. In particular in the field of peritoneal dialysis, hemodialysis, hemofiltration and related methods, the determination of the air volume fraction in the fluids transported is inevitable, in order to achieve an exact dosage of the fluid. In the field of dialysis, the dialysis fluids used for example are composed of a multitude of substances whose type and quantity must be adapted to the requirements of an adequate and individually adjusted patient treatment. The essential tasks of a dialysis apparatus include the delivery with exactly predeterminable dosing rates as well as the quantitative detection of the quantity delivered for balancing purposes.

To achieve an exact dosage, EP 0 941 404 B2 has proposed a pumping device for delivering, balancing and dosing fluids, in particular medical fluids such as blood or dialysis fluid. There is proposed a piston diaphragm pump in which in particular the pump chamber pressure and the stroke volume of the pump are determined as operating parameters. The two parameters can be determined indirectly. For this purpose, in particular a drive pressure of the piston diaphragm pump corresponding to the pump chamber pressure can be measured and a path defining the stroke volume or a change in position of the mechanical delivery means of the pump can be detected.

From DE 199 19 572 A1 a method and an apparatus for calculating the gas content in a fluid, such as blood or dialysis fluid, transported for example through an aforementioned pump has become known already. In this document, it has already been proposed that a measuring means especially provided for the gas content determination can completely be omitted, wherein use is directly made of the pumping, balancing and dosing device employed for example in the dialysis. Corresponding to DE 199 19 572 A1, the fluid in which a corresponding gas is contained therefore both is delivered by the pump and is subjected to a change in volume and pressure. The operating parameters of the pump, which represent the change in volume and pressure, are detected and the gas content is determined from the operating parameters of the pump.

Corresponding to DE 199 19 572 A1, the change in pressure of the fluid/gas mixture preferably is obtained in that the mixture is compressed or decompressed to a predetermined pressure value, i.e. is brought to a predetermined excess or negative pressure. In a first step, a starting pressure of the mixture is detected, in a second step a volume quantity enclosed by closing the inlets and outlets of the pump chamber is created, and in a third step the enclosed volume quantity is subjected to an end pressure different from the starting pressure.

In doing so, the end pressure and the occurring change in volume of the enclosed mixture quantity are detected. The basis for the determination of the gas content is Boyle's Law, according to which the pressure of a gas is increased with constant temperature, when the gas is compressed to a smaller volume. Since the fluid now virtually is incompressible, the change in volume of a fluid/gas mixture following a compression must be based on the compression of the gas contained therein.

To be able to apply the aforementioned principles, however, the system compressibility of the entire pump system must be taken into account, since otherwise on approaching the corresponding pressure levels the gas content back-calculated from the corresponding system parameters can be distorted. For example when using a hydraulically driven piston diaphragm pump, this system compressibility results from the spring properties of the air cushion, the air enclosed in the hydraulic system and through the hydraulic hoses.

It is the object of the invention to improve a generic method or a corresponding apparatus for the determination of gas in a fluid pumped through a pumping device to the effect that the system compressibility of the entire pumping device also is correctly taken into account, in order to be able to perform an exact quantitative determination of the air content in the fluid to be delivered.

In accordance with the invention, this object is solved in a generic method by the combination of the features of claim 1. In the generic method for the determination of gas in a fluid, in which the mixture of gas and fluid both is delivered by a pump and is subjected to a change in volume and pressure, operating parameters of the pump, which represent the change in volume and pressure, are detected, and from the operating parameters of the pump the gas content is determined in consideration of a system compressibility, wherein the system compressibility in the pumping device filled with gas according to the invention is determined by the following steps:
adjusting a starting pressure by means of a pressure sensor,
recording the pump position and the pressure sensor values,
approaching a second pressure level,
recording the pump position and the pressure sensor values in this second position,
determining the spring constant on the basis of the value pairs and equating the spring constant thus determined with the system compressibility.

The system compressibility preferably determined in a machine during its upgrading phase now can be used in the determination of the air content in the fluid delivered, which is already known per se from DE 199 19 572 A1. This procedure offers the advantage to exactly determine the possible air quantity of the fluid, wherein here not a threshold value like in the prior art, but an absolute value can be determined. When balancing the fluid to be delivered, a compensation thus can be effected via the absolute value of the air quantity.

In use of this method during the peritoneal dialysis, the particular advantage is obtained that the discard volume of the bag fluid used for dialysis is reduced.

Particularly advantageous aspects of the invention can be taken from the subclaims following the main claim.

Accordingly, a diaphragm pump, in particular a piston diaphragm pump, can be used as pump.

In this pump, the pump chamber pressure and the stroke volume advantageously are determined as operating parameters.

In accordance with an advantageous aspect of the invention, the piston of the piston diaphragm pump can be driven pneumatically.

In accordance with another advantageous aspect of the invention, the piston of the piston diaphragm pump can be driven hydraulically.

When using a hydraulic drive, air can be accumulated in the hydraulic fluid due to the pump movements and leakages in the hydraulic system, whereby the system compressibility of the entire system is deteriorated. To reduce the system compressibility, the hydraulic fluid therefore is deaerated regularly. The results of the system compressibility determined according to the invention advantageously allow to determine the quality of the degassing operation. As a result, both the intensity and the duration of the degassing operation can be optimized.

In accordance with a further aspect of the invention, the gas can be withdrawn from the piston diaphragm pump via the degassing valve upon reaching a limit value of the gas in the hydraulic fluid.

In accordance with another aspect of the invention an apparatus for performing the aforementioned method is created, in which a detection means for detecting the operating parameters of the pumps and an evaluation unit connected with the detection means is comprised. This evaluation unit serves for evaluating the operating parameters and for determining the gas content in the fluid based on the operating parameters.

In the aforementioned apparatus, the pump preferably constitutes a diaphragm pump, in particular a piston diaphragm pump.

Furthermore, the pump comprises a piston/cylinder unit, a pump chamber defined by a diaphragm, and a pneumatic or hydraulic circuit connected between the piston/cylinder unit and the pump chamber.

Preferably, the pump chamber is divided by the diaphragm into a first chamber, which is in fluid connection with the pneumatic or hydraulic circuit, and a second chamber through which the mixture is delivered.

Furthermore, the apparatus includes a correction unit which adds up the respectively determined gas content of the mixture and settles the same with the entire delivery volume for determining the pure fluid delivery rate. In this way, a dialysis treatment, for example a peritoneal dialysis treatment, can be improved in a particularly advantageous way.

Finally, the invention comprises a dialysis machine, preferably a peritoneal dialysis machine, which includes an apparatus for performing the above-described method for the determination of gas in a fluid pumped through a pumping device.

Further features, details and advantages of the invention will be explained in detail with reference to an embodiment illustrated in the drawing,

The only FIGURE shows a purely schematic representation of an apparatus according to the invention, in which the method according to the invention can be employed.

As is depicted in the schematic representation according to the FIGURE, the pump device is designed as piston diaphragm pump 10 which includes a piston/cylinder unit 12, a pump chamber 14 and a hydraulic circuit 16 connecting the same.

In a manner not shown here in detail, the piston/cylinder unit 12 is driven by a pump drive which acts on the piston 18 of the piston/cylinder unit 12 and moves the same in the cylinder 20. The distance covered by the piston 18 in the cylinder 20 is detected and measured by a non-illustrated length sensor associated to the piston/cylinder unit 12.

The pressure side of the piston/cylinder unit 12 directly is in fluid connection with the hydraulic circuit 16, which acts as means for transmitting the adjusting movement of the piston 18 to the pump chamber 14. The pump chamber 14 comprises a first machine-side chamber 22 which is in fluid connection with the hydraulic circuit 16 and thereby is connected with the pressure side of the piston/cylinder unit 12. The first chamber 22 is defined by a diaphragm 24 which with a corresponding change of the hydraulic volume in the first chamber 22 elastically bulges to the outside in a convex manner or is elastically drawn in towards the interior of the first chamber 22 in a concave manner.

The pump chamber 14 furthermore comprises a second chamber 26 which serves as the actual, volume-variable pump chamber for the medical fluid to be delivered, i.e. for example the dialysis solution 36. The second chamber 26 is formed as head piece which can be placed on the first chamber 22 by means of corresponding fastening means. The disposable chamber 26 has an elastic wall, in particular a diaphragm (not shown here), with which it is placed on the diaphragm 24 in the first chamber 22, so that the two diaphragms come to lie directly one on top of the other. On the second chamber 26 formed as disposable article, a shut-off valve 28 is provided in the delivery line connected with the chamber, by means of which the dialysis solution 38 first is sucked into the second chamber 26 and then can be discharged through a further non-illustrated delivery line upon being pressurized by correspondingly switching over the shut-off valve 28.

The change in volume of the second chamber 26, which is required for fluid delivery, is accomplished by correspondingly actuating the piston/cylinder unit 12. By actuating the piston 18, the hydraulic fluid of the hydraulic circuit 16 is pressed into the first chamber 22 or sucked off from the same. As a result, the diaphragm 24 is actuated, whose movement is transmitted to the second chamber 26 and changes the same in terms of its volume. The illustrated piston diaphragm pump 10 has the great advantage that the corresponding fluid can be delivered very accurately in terms of quantity and the total quantity delivered can be balanced very precisely.

In such piston diaphragm pump, however, air can be accumulated in the hydraulic fluid of the hydraulic circuit 16 due to the pump movements and leakages in the hydraulic system. In the FIGURE, a corresponding air cushion in the hydraulic circuit is designated with reference numeral 34. On the other hand, air can also be accumulated in the dialysis solution, which in the FIGURE is schematically designated with 32. Due to the two air cushions 34 and the air 32 accumulated in the dialysis solution a system compressibility is obtained. Hydraulic hoses also provide a further contribution to the compressibility of the system.

The compressibility of the solution 36, which in the final analysis is measured to determine the gas content in the solution 36, hence is composed altogether of the system compressibility and the air 32 in the solution.

For detecting the gas content in the pump, as described already in DE 199 19 572 A1, a measuring phase is interconnected at each stroke during the pumping operation. At atmospheric pressure, dialysis fluid first is sucked e.g. from a dialysate bag into the second chamber 26, in which an air volume is contained. In a first condition of the apparatus, the starting pressure is measured, which for example is indicated by the fluid column of the dialysate bag. Thereupon, the shut-off valve 28 is closed, whereby a fluid volume enclosed in the second chamber 26 is created. With closed shut-off valve 28 the piston/cylinder unit 12 is actuated, in order to pressurize the enclosed fluid volume in the second chamber 26 with an end pressure. This process takes approximately 0.5 sec. Due to the increase in pressure, the fluid enclosed in the chamber 26 is compressed from a starting volume into an end volume corresponding to its gas content. In this compressed condition, the pressure corresponding to the end pressure again is detected in the hydraulic circuit by a pressure sensor 30. The displacement of the piston 18 taking place during the compression likewise is detected by the length sensor not illustrated here in the FIGURE. With known piston area the volume difference each occurring during the compression can be determined. By determining the dialysis fluid pressure, the tension of the diaphragm 24 can be neglected, as at the time of measurement the diaphragm preferably is in a relaxed position.

From the values obtained by these method steps, a non-illustrated central control device calculates the gas quantity present in the dialysis fluid, i.e. the actual gas volume at atmospheric pressure.

For this purpose, the control unit employs Boyle's Law, which for an isothermal change of state, i.e. by neglecting a change in temperature, reads as follows:

p×V=constant

From the respective starting and end pressures the actual gas volume at atmospheric pressure thus can be calculated. More details can be found in the description of DE 199 19 572 A1.

For the quantitative calculation of the gas volume the system compressibility introduced above must be taken into account. The present invention for the first time provides for distinguishing between the properties of the dialysis fluid on the one hand and the system properties on the other hand. For detecting the system compressibility the following sequence of steps is performed already in the upgrading phase of the pumping device filled with gas:
adjusting a starting pressure by means of a pressure sensor,
recording the pump position and the pressure sensor values,
approaching a second pressure level,
recording the pump position and the pressure sensor values in this second position,
determining the spring constant on the basis of the value pairs and equating the spring constant thus determined with the system compressibility.

As set forth above, each pump stroke must be evaluated for its air content. This is accomplished by the above-described method for air detection, i.e. for detecting the gas content of the dialysis solution 36, which provides the solution compressibility as a result. This solution compressibility now can be compensated with the calculated system compressibility value, so that the evaluation of the air detection can be improved in quality.

As a result, the possible air quantity of the dialysis fluid now can accurately be determined in terms of quantity. A quantitative absolute value is measured here and no more threshold value. When balancing the dialysis fluid, a compensation about the absolute value of the air quantity can therefore be effected. The discard volume of the bag fluid thereby is reduced.

Furthermore, the change of the system compressibility over time can be determined. When using a hydraulic circuit 16, for example the change of the air volume in the hydraulic system, which can be accumulated due to the pump movement and leakages in the hydraulic system, can be monitored. During accumulation of a certain air content in the hydraulic system degassing can be effected via a degassing valve of the hydraulic circuit not shown in detail here in the FIGURE, wherein the intensity and the duration of the degassing operation can be optimized by monitoring the system compressibility.

Accordingly, due to the compensation by means of the system compressibility, a higher balancing accuracy initially can be achieved with the method and a corresponding apparatus as described here. Furthermore, the requirements of the pump hardware and the hydraulic system can be reduced, since these properties can be compensated technically.

Furthermore, the degassing quality and the necessity of degassing can be detected and evaluated systematically, which leads to time savings with regard to making the machine ready for operation. Finally, leakages of the hydraulic system are cyclically detected during the course of the treatment. A quality determination of the cassette rinsing operation also is possible during upgrading, which furthermore results in a discard optimization of the dialysate solution.

1. A method for the determination of gas in a fluid, wherein the mixture of gas and fluid both is delivered by a pump and is subjected to a change in volume and pressure, operating parameters of the pump, which represent the change in volume and pressure, are detected, and from the operating parameters of the pump the gas content is determined in consideration of a system compressibility,
characterized in
that the system compressibility in the pumping device filled with gas is determined by the following steps:
adjusting a starting pressure by means of a pressure sensor,
recording the pump position and the pressure sensor values,
approaching a second pressure level,
recording the pump position and the pressure sensor values in this second position,
determining the spring constant on the basis of the value pairs and equating the spring constant thus determined with the system compressibility.

2. The method according to claim 1, characterized in that a diaphragm pump, in particular a piston diaphragm pump, is used.

3. The method according to claim 2, characterized in that as operating parameter the pump chamber pressure and the stroke volume is determined.

4. The method according to claim 3, characterized in that the piston of the piston diaphragm pump is driven pneumatically.

5. The method according to claim 4, characterized in that the piston of the piston diaphragm pump is driven hydraulically.

6. The method according to claim 5, characterized in that the gas content of the hydraulic fluid driving the piston diaphragm pump is determined.

7. The method according to claim 6, characterized in that upon reaching a limit value the gas is withdrawn from the piston diaphragm pump via a degassing valve.

8. An apparatus for performing claim 1 for the determination of gas in a fluid of a pump for the delivery as well as change in volume and pressure of the mixture of gas and fluid, a detection means for detecting operating parameters of the pump, and an evaluation unit connected with the detection means for evaluating the operating parameters and for determining the gas content in the fluid from the operating parameters.

9. The apparatus according to claim 8, characterized in that the pump constitutes a diaphragm pump, in particular a piston diaphragm pump.

10. The apparatus according to claim 9, characterized in that the pump includes a piston/cylinder unit, a pump chamber defined by a diaphragm, and a pneumatic or hydraulic circuit connected between the piston/cylinder unit and the pump chamber, wherein preferably the pump chamber is divided by the diaphragm into a first chamber, which is in fluid connection with the pneumatic or hydraulic circuit, and a second chamber through which the mixture is delivered.

11. The apparatus according to claim 1, wherein a correction unit is provided, which adds up the respectively determined gas content of the mixture and settles the same with the entire delivery volume for determining the pure fluid delivery rate.

12. A dialysis machine, preferably a peritoneal dialysis machine, comprising an apparatus according to claim 1 for performing the method according to claim 1, characterized in that the gas content in the pumped fluid is determined at every pump stroke.
